(19) United States
(12) Patent Application Publication
(10) Pub. No.: US 2004/0081638 A1
(43) Pub. Date: Apr. 29, 2004

(54) Delivery of disease control in aquaculture and agriculture using nutritional feeds containing bioactive proteins produced by viruses

(76) Inventor: David J. Kyle, Catonsville, MD (US)

Correspondence Address: Finnegan, Henderson, Farabow,, Garrett & Dunner, L.L.P., 1300 I Street, N.W., Washington, DC, 20005-3315, US

(21) Appl. No.: 10/683,361

(22) Filed: Oct. 14, 2003

Related U.S. Application Data
(63) Continuation-in-part of application No. PCT/US02/27198, filed on Aug. 27, 2002.
(60) Provisional application No. 60/314,637, filed on Aug. 27, 2001.

(51) Int. Cl. A61K 48/00; A23K 1/165; A23K 1/17

(52) U.S. Cl. 424/93.2; 424/442

(57) ABSTRACT

An animal feed is provided with a macroalgal, plant, or animal, e.g., insect or crustacean, biomass with one or more non-native peptides, proteins, antibodies, therapeutics, or a combination thereof. The proteins can be therapeutic, bioactive, proteins. A gene encoding a protein, antibody, therapeutic, or combination thereof, can be incorporated into a virus, which in turn, infects an organism that is a component of the feed. The virus can infect the macroalgal, plant, or animal feed component without incorporating viral genes into the macroalgal, plant, or animal feed component.

### CROSS-REFERENCE TO RELATED APPLICATIONS

This application is a Continuation-In-Part of PCT/US02/27198, filed Aug. 27, 2002, which claims the benefit of U.S. Provisional Application No. 60/314,637, filed Aug. 27, 2001, the benefit of the filing dates of which are claimed, and the disclosures of which are incorporated by reference in their entireties.

### BACKGROUND OF THE INVENTION

1. Field of the Invention

This invention is directed to edible materials, including feeds, feed additives, and therapeutics, that are components of animal feeds used in aquaculture or in agriculture. These edible materials contain exogenous peptides, proteins, and/or antibodies and their fragments, that can convey resistance or immunity to viral or bacterial pathogens, or otherwise improve the health and performance of the species that consume them. The exogenous peptides, proteins, and/or antibodies and their fragments can be expressed inside the edible materials by infecting the edible material with a recombinant virus that encodes the exogenous peptides, proteins, and/or antibodies and their fragments.

2. Related Art

Plant products have been produced using specific genetic modification to express proteins and/or antibodies of therapeutic value. The group at the Boyce Thompson Institute at Cornell has cloned a viral coat protein into bananas capable delivering an oral vaccine when ingested by humans. However, as yet, this concept has not been extended to microbes.

There are several plant biotech companies such as Meristem, Large Scale Biology, and Prodigene, which are now expressing certain human therapeutic proteins, including antibodies, in plants. Large Scale Biology is expressing proteins in tobacco plants using a tobacco mosaic virus as a vector to produce the protein of interest. The protein is then isolated and purified from the plant material and used for human therapeutic purposes. In this way the plant genome itself is actually not modified, but rather the genome of the infecting virus carries the gene of interest.

Recombinant microbes, including bacteria, yeast, and filamentous fungi, have been used to produce human therapeutic proteins. However, such recombinant microbes have not been used in aquaculture or agriculture, wherein the cultivated animal ingests the whole organism. Rather, to date, the recombinant organism has been used as a factory from which the therapeutic protein is isolated and purified prior to use.

Certain plant products have been produced that contain proteins and/or antibodies of therapeutic value by infecting the plant with a virus that expresses the protein of interest. Large Scale Biology has a series of patents protecting this technology, but its purpose is to produce purified proteins for pharmaceutical purposes, which requires an extensive purification procedure following harvest of the plant material. These patents do not involve the use of the intact plant material as a source of both nutrition and disease control, except under the unusual condition that the pharmaceutical product is expressed in the fruit of the plant.

Certain recombinant proteins have been produced in insect cells using an insect virus expression system (baculovirus). These proteins are also produced in intact insect larvae following infection with modified baculoviruses. In both cases, the insect cells or larvae are used as factories to produce the protein of interest, and the recombinant protein is then purified for pharmaceutical purposes. Insect cells or larvae infected with baculovirus are particularly useful in the expression of certain human therapeutic proteins because the post-translational modifications of the therapeutic proteins are similar to the post-translational modifications imparted upon expression in human cells.

The Sindbis arbovirus can be used to deliver high levels of gene expression in vivo in non-host arthropod species without causing cytopathic effects in infected cells or impairing the development of the organism. A replication-competent Sindbis virus, containing the coding region of green fluorescent protein (GFP), produced productive infections when injected into insect larvae and pupae (Lewis, et al., 1999). Thus, virus-mediated ectopic gene expression has been accomplished in arthropods, a phylum that includes the classes Crustacea and Insecta.

Antibiotic doping is used routinely in the aquaculture setting. Typically, the pure or semipure antibiotics are added directly to the water column. However, neither crude fermentation broths nor crude preparations including cells have been used for any kind of therapeutic delivery system.

Production of amino acids, such as lysine, typically involves a genetically modified microorganism, which overproduces the amino acid of interest and excretes it into the fermentation medium. The wastestream from such a fermentation would include biomass containing the amino acid, and this wastestream product could be used as a crude delivery form of the small molecule nutritive amino acid.

A baculovirus expression system is an efficient method for expressing proteins in insect cell culture. Baculovirus is in the family Baculoviridae, a diverse group of large double stranded DNA (dsDNA) viruses that infect arthropods, including insects, arachnids, and crustaceans. Baculoviruses are species-specific and do not infect vertebrates, nor can they propagate in mammalian cells in culture.

Fungi, such as yeast, and bacteria are also in the direct food chain of fish, crustaceans, and mollusks. However, only a few of these microbes, perhaps less than 10 species, have been exploited for aquaculture feeds. These few species have been used primarily for historical reasons and ease of cultivation. They have not been chosen on the basis of any scientific evidence of superiority as nutritional or therapeutic supplements.

The marine environment is filled with bacteria and viruses that can attack fish and shellfish, thereby devastating aquaculture farms very quickly. Bacteria and viruses can also attack single-celled microalgae, so these organisms have evolved biochemical mechanisms to defend themselves from such attacks. Such mechanisms may involve the secretion of compounds that inhibit bacterial growth or viral attachment.

### SUMMARY OF THE INVENTION

The present invention provides a feed, feed additive, and therapeutic, and the use of such feed, feed additive, and therapeutic to deliver a therapeutic dose of a bioactive peptide or protein. The invention also provides a method of feeding the feed, feed additive, and therapeutic to animals cultivated in agriculture and aquaculture.

In one embodiment, this invention provides an aquaculture or an agriculture feed containing plant biomass comprising one or more proteins, antibodies, or a combination thereof, where the proteins and antibodies are non-native to the plants. Preferably, the host plants are selected from tobacco, corn, soybean, canola, sunflower, or any other cultivated crop. The plant genome itself can be modified to express the proteins or antibodies or antibody fragments. Alternatively, the plants are infected with a virus or viruses, which encode the proteins or antibodies or antibody fragments recombinantly. While in some cases, the host and expressed protein may be consumed together without further processing, preferably the entire plant material, not only the fruit, would be modified in some way to make the material edible to non-human animals. Such a modification can include, but is not limited to, homogenizing, cooking, baking, extruding, solubilizing, or treating with enzymes.

In another embodiment, this invention provides an aquaculture or an agriculture feed containing insect biomass comprising one or more proteins, antibodies, or a combination thereof, where the proteins and antibodies are non-native to the insects. Preferably, the insects are larval stages of lepidoptera. The insect genome itself can be modified to express the proteins or antibodies or antibody fragments. Alternatively, the insects are infected with a virus or viruses, which encode the proteins or antibodies or antibody fragments and are also expressed recombinantly upon infection. In a preferred mode, the insect material would be modified in some way to make the material edible to non-human animals. Such a modification can include, but is not limited to, homogenizing, cooking, baking, extruding, solubilizing, or treating with enzymes. This invention contemplates the use of the whole insect larvae, or a portion thereof, as a feed additive. This invention also contemplates the use of the larvae along with its entire larval cultivation matrix, as all these materials may convey feed materials. Such a larvae will typically contain the protein or proteins of interest, but purification steps are not necessary for its use in animal feeds.

In a further embodiment, this invention provides an aquaculture feed, agriculture feed, or human food containing a macroalgal biomass comprising one or more peptides, proteins, antibodies, or a combination thereof, where the peptides, proteins, and antibodies are non-native to the algae.

In yet another embodiment, this invention provides a method of delivering therapeutic proteins or peptides to a non-human animal by administering a feed comprising one or more algae (e.g., macroalgae), plants, or arthropods (e.g., crustaceans or insects) expressing a non-native therapeutic protein. This method is particularly suitable for the non-human animal in agriculture or for fish and shellfish in aquaculture. In a preferred mode, the therapeutic peptide, peptides, protein or proteins is (are) recombinant protein(s) expressed directly by the plant or insect. Alternatively, the algae, plants, arthropods, or other animals are infected by a recombinant virus, which expresses the therapeutic protein recombinantly.

Preferred therapeutic proteins include a peptide, peptides, protein, or proteins that inhibit(s) the growth or replication of a pathogen, such as a Vibrio species, or a protein or proteins that inhibit(s) shrimp viruses, such as, but not limited to, Taura or White spot virus infection in shrimp, or recombinantly expressed antibody or antibody fragments to pathogens, such as bacteria or viruses, or a protein that, when introduced orally to an animal, will immunize the animal, as in the case of an oral vaccine.

In a further embodiment, this invention provides a method of transfecting or infecting crustaceans with non-native therapeutic proteins using baculovirus. This method is particularly suitable for crustaceans in aquaculture. Preferably, the crustaceans are Pacific white shrimp (Penaeus vannamei) and the baculovirus is Autographa californica nuclear polyhedrosis virus (AcNPV). The crustacean can be infected either by injection or orally by incorporating the virus into the crustacean's food. The baculovirus can be engineered to express green fluorescent protein (GFP) for monitoring infection. For example, the therapeutic proteins can inhibit the growth or replication of bacteria (e.g., Vibrio) or viruses (e.g., Taura or White Spot virus).

### DESCRIPTION OF THE DRAWING

FIG. 1. Pacific white shrimp (Penaeus vannamei) were transfected orally with an engineered baculovirus (AcNPV-eGFP) to express green fluorescent protein (GFP) as a fusion protein. A 720 kb fragment containing GFP was fused to the polyhedron (polh) promoter and flanked by Xho I sites 3′ to polh. The Bacmid Bac-to-Bac® Baculovirus Expression system (Invitrogen) was utilized for cloning and transfection. Transfected cells or purified virus were combined with shrimp food and fed to shrimp. Seventy-two hours after consuming the virally infected feed, the shrimp were placed in a petri dish and observed on a Dark Reader® transilluminator (Claire Chemical Research). Shrimp expressing GFP exhibited a greenish glow located specifically within the hepatopancreas area in the cephalothorax. Uninfected shrimp demonstrated no fluorescence. Further detail is provided in Example 7.

### DETAILED DESCRIPTION OF THE INVENTION

Definitions

A “feed” is a preparation providing nutritional value to any animal, including, but not limited to, terrestrial animals (e.g., humans, cattle, horses, pigs, sheep, goats, and poultry) and aquatic animals (e.g., fish, shrimp, lobsters, crawfish, mollusks, sponges, and jellyfish).

A “feed additive” is any substance added to feed, regardless of nutritional or therapeutic value.

A “therapeutic” is a substance that can heal, or provide a remedial, palliative, or preventive effect on a pathologic process. Therapeutic substances and compounds can be used to treat medical diseases, disorders, conditions, or syndromes.

“Macroalgae” refers to algae that form structures easily discernable with the naked eye in at least one life stage. Usually these organisms have secondary vascularization and organs. Examples of different groups containing macroalgae include, but are not limited to, the chlorophyta, rhodophyta, and phaeophyta.

“Microalgae” include both prokaryotic and eukaryotic algae that are classed in many different genera. Prokaryotic algae are typically referred to as cyanobacteria or bluegreen algae. Eukaryotic microalgae come from many different genera, some of which overlap with the macroalgae, but can be generally differentiated by their size and lack of defined organs. Microalgae can have specialized cell types. Examples of different groups containing microalgae include, but are not limited to, the chlorophyta, rhodophyta, phaeophyta, dinophyta, euglenophyta, cyanophyta, prochlorophyta, and cryptophyta.

An “antibiotic” is a substance that can inhibit or stop the growth of microorganisms or that can kill microorganisms.

“Bacteriocidal” refers to the ability to kill bacteria. “Bacteriostatic” refers to the ability to inhibit or stop the growth of bacteria.

An “immunogenic epitope” is a discrete site of an antigenic molecule against which an antibody will be produced, to which the T-cell receptor responds, an antibody binds, or otherwise induces any other immune response.

“Passive immunity” is immunity conveyed by molecules, e.g., antibodies, immunogens, other proteins, or sensitized lymphocytes that deliver protection from antigens, and that are obtained from a source outside an organism's own immune system. Passive immunity can be acquired by an oral route, e.g. from an organism, antibody, or other molecule that enters the gastrointestinal system and provides immunity (e.g., by preventing infestation across the gastrointestinal mucosa) or by stimulating the gastrointestinal immune system (e.g., IgA antibodies, or gut-associated lymphoid tissue (GALT)). Passive immunity can also be acquired by the transfer of antibodies from one animal to another (e.g., the passive immunity an offspring acquires from its mother).

“Aquaculture” is the cultivation of aquatic organisms under controlled conditions. An “aquatic organism” is an organism grown in water, either fresh- or saltwater. Aquatic organisms, include, but are not limited to, fish, e.g., bass, striped bass, tilapia, catfish, sea bream, rainbow trout, zebrafish, red drum, and carp; crustaceans, e.g., penaeid shrimp, brine shrimp, freshwater shrimp, and Artemia; and rotifers.

“Probiotic” refers to the promotion of the growth of an organism. Probiotic effects, e.g., therapeutic or protective effects, can be delivered by probiotic organisms. Probiotic organisms include algae, bacteria, and fungi, such as yeast.

A “patient” is any living animal, including, but not limited to, a human, who has, is susceptible to, or is suspected of having or being susceptible to, a pathologic condition, disease, disorder, or syndrome who otherwise would be a subject of investigation relevant to a pathologic condition, disease, disorder, or syndrome. Accordingly, a patient can be an animal that has been bred or engineered as a model for any pathologic condition, disease, or disorder. Similarly, a patient can be an animal (such as a farm animal, dairy animal, ranch animal, animal that lives under water, animal cultivated on land or in water for food or other commercial use, an experimental animal, or a pet animal) including a human, who is serving as a healthy control for investigations into pathologic conditions, diseases, disorders, or syndromes.

Detailed Description of Various Embodiments of the Invention

Viral or bacteria infections can dramatically limit farm productivity in terrestrial environments. The marine environment is also filled with bacteria and viruses that can attack fish and/or shellfish. Infection by bacteria or viruses can devastate intensive marine-based farms very quickly. For example, one of the major disease control problems in shrimp aquaculture today is infection by certain viruses (e.g., White Spot, Taura, etc.). Conventional strategies (e.g., antibiotics) are not effective in this situation, and shrimp cannot be vaccinated by methods analogous to those used for fish. Shrimp, like all crustaceans, have only a rudimentary immune system, so they are particularly susceptible to devastation by viral attacks.

This invention provides a solution to this problem with a biological control method using the target animal's feed, for example, algae, plants, arthropods, and animals, as the vector to deliver therapeutic peptides or proteins (e.g., antibodies) directly to the target animal (e.g., shrimp). Such “designer feeds” can be a normal part of the diet and can modified to deliver a therapeutic dose of antibody directly to the shrimp's gastrointestinal system. This provides passive immunity: the exogenous antibody remains outside the host organism and prevents infestation through the gut wall. The invention envisions the use of organisms such as transgenic algae, plants, and animals, (e.g., arthropods) to deliver the antibody to the virus. Such probiotics, as envisioned in the invention, do not have to replicate in the target organism for the desired effect to occur. Alternatively, the organism itself can be infected with a virus engineered to produce the antibody of interest. Alternatively, the organism can deliver a portion of the virus (e.g. a coat protein or coat proteins) or fragment thereof, in order to immunize a terrestrial or aquatic animal, such as a fish or shrimp.

This invention provides advantages in production and delivery of the antimicrobial compounds or antibodies by packaging them in a plant or insect source compared to a fermentative source. Sterile fermentation is important for FDA compliance in terms of drug good manufacturing practice (Drug-GMP), whereas the less-pure sources from plants or insects will suffice for animal use. Plants and insects are common elements in the food chain of both aquatic species in aquaculture and terrestrial species in agriculture. A second value in eukaryotic processing (e.g., by plants or insect cells) is that the post-translational modifications undertaken by these species may be more “native” compared to recombinant products from bacteria. One of the major problems with bacterial production of human proteins is that the microbially produced recombinant proteins can be ineffective because of incorrect post-translation modifications.

Autographica californica nuclear polyhedrosis virus (AcNPV) is a baculovirus commonly used in laboratory protein expression. It is shed from cells during early stages of infection by budding of the cell membrane; in later stages of infection, viruses are encased in intracellular occlusion bodies, which are large protein crystals. AcNPV is commonly used in the laboratory to infect insect cell culture lines. Vectors and molecular biology supplies, as well as methods for baculovirus expression vector systems, including AcNPV, are readily available from commercial suppliers.

Antibodies or antibody fragments to desired targets, such as White Spot virus or Taura virus, can be prepared by routine techniques (e.g., immunization and selection of monoclonal antibody producing hybridomas) or by screening viral or bacterial expression libraries of immunoglobulin genes and gene fragments (Coligan, et al.). Nucleic acid sequences encoding the binding sites of the selected antibodies can be cloned using standard methods (Ausubel, et al.) and antibodies can be expressed from recombinant algae, plants, arthropods, or other animals, or cloned into viruses that infect the desired feed materials.

There are a number of well known bactericidal and bacteriostatic peptides that inhibit microbial growth. These include, but are not limited to, cecropins, penaeidins, bactenecins, callinectins, myticins, tachyplesins, clavanins, misgurins, pleurocidins, parasins, histones, acidic proteins, and lysozymes. These peptides can be made in a plant, such as tobacco, soybean, corn, sunflower, cotton, safflower, canola, or any other agronomic species using recombinant methods well known to those in the art, and thus provided as a feed component to convey resistance or tolerance to infestation. Suitable plant material also includes macroalgae (e.g., kelps), which are grown worldwide as a commodity feed crop in aquaculture. Macroalgae are the foodstuffs of many aquaculture species, and this invention contemplates recombinant production of therapeutic proteins in the natural or farm diet of juvenile fish (e.g., half-grown catfish), as well as fish larvae. Thus, within the contemplation of this invention are macroalgae, insects, or other host organisms that make up part of the food chain for the feeding of larvae, juveniles, and adults in aquaculture, as well as the food chain for a similar life sequence in terrestrial animals (e.g., pigs, chickens, and cows).

Edible materials (i.e., any materials that can be ingested) are preferably of microbial, plant or animal (vertebrate or invertebrate) origin. Edible materials can comprise the whole plant or animal, or any parts thereof. The invention includes genetically modified plants and animals that produce the exogenous protein, peptide, antibody and/or antibody fragments directly.

Post-harvest processing can be performed to further prepare the material for use as feeds. This invention contemplates conventional (known) processes for converting macroalgal, insect, or plant material into feeds. Such conventional processes include homogenization followed by extrusion into pellets of various sizes, depending on the application (e.g., larval, juvenile or adult). Other modes of preparation include spray drying, fluid bed drying, or providing the material as a liquid suspension.

The invention provides a feed, feed additive, or therapeutic that contains an organism, or any part of an organism, that comprises one or more proteins, peptides, antibodies, antibody fragments, or combination thereof, which are non-native to the organism and produced by a recombinant virus infecting the organism.

The invention provides that the organism is an alga (e.g., a macroalga or a microalga). The organism also can be an animal (e.g., an animal raised in agriculture or aquaculture). The invention also provides that the organism can be a yeast or bacterium (e.g., Phaffia or Lactobacillus). The organism can be an arthropod, such as an insect or a crustacean. The crustacean can be a shrimp. The organism also can be a plant (e.g., an agronomic plant such as tobacco, soybean, corn, sunflower, cotton, safflower, or canola).

The invention further provides that the protein, peptide, antibody, antibody fragment, or combination thereof is produced by a recombinant virus infecting the organism. The protein, peptide, antibody, antibody fragment, or combination thereof, is specific for bacteria or viruses causing disease in the respective animal cultivated in water, farm animal, ranch animal, dairy animal, pet animal, or human patient.

The invention yet further provides that the protein, peptide, antibody, antibody fragment, or combination thereof, can include cecropins, penaeidins, bactenecins, calinectins, myticins, tachyplesins, clavanins, misgurins, pleurocidins, parasins, histones, acid proteins, and lysozymes.

The invention provides a human food, food additive, or therapeutic which comprises an organism selected from algae, plants, arthropods, or other animals, or parts thereof, comprising one or more proteins, peptides, antibodies, antibody fragments, or combination thereof, which are non-native to the organism and produced by a recombinant virus infecting the organism. The arthropod can be an insect or a crustacean (e.g., a shrimp).

The invention also provides a method of feeding a farm, ranch, dairy, or pet animal by providing a feed comprising an organism, or any part thereof, selected from bacteria, yeast, plants, algae, animals, or crustaceans, comprising one or more proteins, peptides, antibodies, antibody fragments, or a combination thereof that are non-native to that organism; and administering the feed to the animal.

The invention further provides a method of feeding a farm, ranch or dairy animal raised in agriculture. This animal can be a cow, pig, or chicken.

The invention yet further provides a method of feeding an animal a feed comprising an organism, wherein the organism is infected by a virus engineered to produce proteins, peptides, antibodies, or antibody fragments in the organism. The proteins, peptides, antibodies, or antibody fragments can be expressed without incorporating exogenous genes into the organism's genome. The animal can be raised in agriculture, and can be (e.g., a cow, pig, or chicken). The animal can also be raised in aquaculture and can be a crustacean (e.g., a shrimp) or a fish.

The invention provides that the peptide, protein, antibody, or antibody fragment specifically binds to an infectious agent of disease in the farm, ranch, dairy, or pet animal. The invention also provides that the peptide, protein, antibody, or antibody fragment specifically binds to a molecule produced by an infectious agent (e.g., a toxin, such as pertussis toxin).

The invention also provides a method of feeding an animal cultivated in water, by providing a feed comprising an organism, or any part thereof, selected from algae, plants, arthropods, other animals, comprising one or more proteins, peptides, antibodies, antibody fragments, or a combination thereof, that are non-native to that organism; and administering the feed to the animal.

The invention further provides a method of feeding a human or non-human animal, wherein the human or non-human animal is provided with an organism infected with a recombinant virus, derived from a non-vertebrate source, engineered to produce a protein, peptide, antibody, or antibody fragment that is expressed without incorporation into the organism's genome. The peptide, protein, antibody, or antibody fragment can inhibit the growth of Vibrio species in vivo or in vitro, can inhibit viral infection in shrimp (e.g., Taura virus or White spot virus), or can specifically bind to an infectious agent of disease in an animal cultivated in water.

The invention yet further provides that the recombinant virus is a baculovirus (e.g., Autographa californica nuclear polyhedrosis virus (AcNPV)) or an arbovirus (e.g., Sindbis virus).

The invention also provides a method of using a feed, feed additive, or therapeutic, for an animal. The feed, feed additive, or therapeutic contains an organism, or any parts thereof, comprising one or more proteins, peptides, antibodies, antibody fragments, or combination thereof, which are non-native to the organism, and are produced by a recombinant virus infecting the organism. The method can be used for the treatment or prevention of a disease of an aquatic animal, a terrestrial animal, a pet animal, or a human.

The invention further provides a method of using a feed, feed additive, or therapeutic, as a vaccine. The feed, feed additive, or therapeutic contains an organism, or any parts thereof, comprising one or more proteins, peptides, antibodies, antibody fragments, or combination thereof, which are non-native to the organism, and produced by a recombinant virus infecting the organism. The method can be used to vaccinate an aquatic animal, a terrestrial animal, a pet animal, or a human. Accordingly, the invention includes a vaccine and/or immunostimulant.

The invention further provides a method of delivering a protein to an animal by feeding the animal a biomass of an alga, plant, arthropod (e.g., insect or crustacean), or other animal infected with a recombinant virus that expresses the protein. The animal can be a human, aquatic animal (e.g., a shrimp or fish), terrestrial animal (e.g., a farm, ranch, dairy, or pet animal).

In embodiments, the invention provides that the protein is therapeutic (e.g., inhibits the growth or replication of Vibrio, Taura, or White spot).

In embodiments, the protein is an antibody.

In embodiments, the virus is in the family Baculoviridae (e.g., is a nucleopolyhedrovirus such as an Autographa californica nuclear polyhedrosis virus). The virus can also be an arbovirus such as a Sindbis virus.

The invention yet further provides a method of delivering a protein to an animal by feeding the animal algal, plant, or arthropod (e.g., insect or crustacean) biomass infected with a recombinant virus that expresses the protein, wherein the animal is an arthropod, such as an insect, or a crustacean, such as a shrimp, (e.g., Penaeus vannamei).

### EXAMPLES

Certain embodiments of the invention will now be described in more detail through the following examples. The examples are intended solely to aid in more fully describing selected embodiments of the invention and should not be considered to limit the scope of the invention in any way.

### Example 1

Incorporation of a White Spot Virus Antibody into a Plant-Based Feed. A particular viral or bacterial pathogen is chosen and used to prepare monoclonal antibodies using procedures described in “Current Protocols in Immunology” or other procedures known to those skilled in this field. The White spot virus, for example, contains three major coat proteins, and antibodies or antibody fragments can be prepared to any or all of these proteins. Gene(s) coding for this antibody or an appropriate antibody fragment (e.g., Fab) are isolated and amplified in an appropriate vector (e.g. Invitrogen's TOPO TA cloning vectors). The gene is spliced into a transformation vector suitable for plant transformation (e.g., pYLTAC7 from Riken Gene Bank). The transformation vector is chosen so that the antibody will be overexpressed in the plant cellular biomass. The vector may be targeted to the edible portion of the plant (i.e., seeds) so that normal harvesting methodologies can be used. Alternatively, the vector may be targeted to the unused portion of the plant (stems and leaves) so that these less valued materials can be used as value added components to the crop plant without affecting the yield or quality of the normally harvested portion. The biomass in which the antibody is expressed is then used as a feed additive in such a way as to provide the antibody or antibody fragment directly to the animal, thus providing passive immunity.

### Example 2

Expression of a Bactericidal or Bacteriostatic Protein in a Plant-Based Feed. A bactericidal or bacteriostatic protein is chosen for the particular application. For example, proteins of the penaeidin class may be chosen for pathogenic control in shrimp. Penaeidins are members of a family of antimicrobial peptides isolated from crustaceans (e.g., Penaeus shrimp). Antimicrobial peptides may also come from insects and chelicerates and may include, but are not limited to, cecropins, penaeidins, bactenecins, callinectins, myticins, tachyplesins, clavanins, misgurins, pleurocidins, parasins, histones, acidic proteins, and lysozymes. The gene for the chosen protein or peptide is either isolated from the original source, an amplification source, or it can be made synthetically. The gene is spliced into a transformation vector suitable for plant transformation. The transformation vector is chosen so that the antibody will be overexpressed in the plant cellular biomass (e.g., tobacco leaves or potato tubers). The vector may be targeted to the edible portion of the plant (i.e., seeds) so that normal harvesting methodologies can be used. Alternatively, the vector may be targeted to the unused portion of the plant (stems and leaves) so that these materials can be used as value added components to the crop plant without affecting the yield or quality of the normally harvested portion. This biomass is then used as a feed additive in such a way as to provide the bactericidal protein directly to the animal thus providing resistance to that particular pathogen.

### Example 3

Incorporation of a Gene for an Antibody or Antibody Fragment into a Plant-Based Virus and Use of the Infected Plant Material as Feed. A particular viral or bacterial pathogen is chosen and used to prepare monoclonal antibodies using procedures described in “Current Protocols in Immunology” or other procedures known to those skilled in this field. Gene(s) coding for this antibody or an appropriate antibody fragment (Fab) are isolated and amplified in the appropriate vector. The gene is spliced into the genome of a selected plant virus such as tobacco mosaic virus (TMV), alfalfa mosaic virus (AMV), or cauliflower mosaic virus (CMV). This recombinant virus is then used to infect a plant (mature or seedling). As the virus replicates in the plant material, it will express the antibody or antibody fragment directly in the plant material. The entire plant can then be harvested and used directly as feed material. Alternatively, the plant material may be homogenized and extruded into pellets suitable for feed applications. The viruses should not be a concern in feeding, since they will not infect the animals consuming the feed, but to the extent there is a concern, they can be inactivated by high temperature or other procedures familiar to those in the field prior to use of the plant material as feeds.

### Example 4

Incorporation of a Gene for a Bactericidal or Bacteriostatic Protein into a Plant-Based Virus and Use of the Infected Plant Material as Feed. A bactericidal or bacteriostatic protein is chosen for the particular application. For example, peptides of the penaeidin class may be chosen for pathogenic control in shrimp. Penaeidins are members of a family of antimicrobial peptides isolated from crustaceans (e.g., Penaeus shrimp). Antimicrobial peptides may also come from insects and chelicerates and may include but are not limited to cecropins, penaeidins, bactenecins, callinectins, myticins, tachyplesins, clavanins, misgurins, pleurocidins, parasins, histones, acidic proteins, and lysozymes. The gene for the chosen protein or peptide is either isolated from the original source, an amplification source, or it can be made synthetically. The gene is spliced into the genome of a selected plant virus such as tobacco mosaic virus (TMV), alfalfa mosaic virus (AMV), or cauliflower mosaic virus (CMV). This recombinant virus is then used to infect a plant (mature or seedling). As the virus replicates in the plant material, it will express the protein directly in the plant material. The entire plant can then be harvested and used directly as feed material. Alternatively, the plant material may be homogenized and extruded into pellets suitable for feed applications. The viruses can be inactivated by high temperature or other procedures familiar to those experts in the field prior to use as feeds.

### Example 5

Incorporation of a Gene for an Antibody or Antibody Fragment into an Insect-Based Virus and Use of the Infected Insect Material as Feed. A particular viral or bacterial pathogen is chosen and used to prepare monoclonal antibodies using procedures well known to those of skill in this field. Gene(s) coding for this antibody or an appropriate antibody fragment (Fab) are isolated and amplified in the appropriate vector. The gene is spliced into the genome of a selected insect virus, such as baculovirus. This virus is then used to infect insect larvae. As the virus replicates in the larval insect, the antibody or antibody fragment will be expressed directly in the larval cells. The entire larvae can then be harvested and used directly as feed material. Alternatively, the larvae may be homogenized and extruded into pellets suitable for feed applications. The viruses can be inactivated by high temperature or other procedures known in this field prior to use as feeds.

### Example 6

Incorporation of a Gene for a Bacteriostatic or Bactericidal Protein into an Insect-Based Virus and Use of the Infected Insect Material as Feed. A bactericidal or bacteriostatic protein is chosen for the particular application. For example, proteins of the penaeidin class may be chosen for pathogen control in shrimp. Penaeidins are members of a family of antimicrobial peptides isolated from crustaceans (e.g., Penaeus shrimp). Antimicrobial peptides may also come from insects and chelicerates and may include but are not limited to cecropins, penaeidins, bactenecins, callinectins, myticins, tachyplesins, clavanins, misgurins, pleurocidins, parasins, histones, acidic proteins, and lysozymes. The gene for the chosen protein or peptide is either isolated from the original source, an amplification source, or it can be made synthetically. The gene is spliced into the genome of a selected insect virus, such as baculovirus. This recombinant virus is then used to infect insect larvae. As the virus replicates in the larvae it will express the protein directly in the larval tissues. The entire larvae can be harvested and used directly as feed material. Alternatively, the larvae may be homogenized and extruded into pellets suitable for feed applications. The viruses can be inactivated by high temperature or other procedures familiar to those in the field prior to use as feeds.

### Example 7

Incorporation of a Gene for a Therapeutic Protein into Baculovirus and the Use of the Infected Material as Feed. Pacific white shrimp (Penaeus vannamei) were transfected orally with an engineered baculovirus (AcNPV-eGFP) to express GFP as a fusion protein. The Bacmid Bac-to-Bac® Baculovirus Expression system (Invitrogen) was utilized for cloning and transfection. A 720 kb fragment containing GFP was fused to the polyhedron (polh) promoter and flanked by Xho I sites 3′ to polh. Using methods described in the Invitrogen product literature, Sf9 insect cells were transfected with the recombinant baculovirus. After 72 hours, plaque formation was visually confirmed, and 70 ml culture fluid medium was pelleted at 100 g for 5 minutes at 4° C. The resulting cell pellet was maintained at 4° C. until it was subsequently used for oral infection. The corresponding resulting supernatant fluid was centrifuged for 2 hours at 80,000 g at 4° C. on a 27% sucrose gradient to yield purified virus. This sucrose-purified virus pellet was maintained at 4° C. until it was subsequently used for oral infection.

Shrimp isolation chambers, consisting of 3-qt containers filled with 30 ppt salinity dechlorinated water, were provided with air stones for oxygenation. Three one-gram shrimp were placed in each container and allowed to acclimatize overnight.

The following procedures were performed within 30 minutes prior to feeding the shrimp. A pellet matrix was prepared by first adding 100 mg of alginic acid (Sigma) to 10 ml of distilled deionized water (ddH₂O) in a beaker and heating to 40° C. while stirring. After the gel began to form, 150 mg of starch (Sigma) was added. The solution was allowed to mix for a minute before addition of 500 mg of krill meal. While continuing to stir the solution, the heat source was removed.

An aliquot of pellet matrix (500 μl) was combined with either 5 μl of the infected cell pellet or 5 μl of sucrose-purified virus, and gently mixed with a vortex mixer. The infected pellet matrix was aspirated into a tuberculin syringe to which a 21-gauge needle was subsequently attached. A formation solution was formed by dissolving 5 grams of calcium chloride (J. T. Baker) and 1 gram of sodium chloride (Research Organics) in 100 ml of ddH₂O. While the formation solution was stirring slowly, the matrix was squeezed through the needle into the solution to form tubular pellets. Pellets formed immediately upon impact in solution and a spatula was used to clean the needle between pellets. Pellets appeared to be 25-30 l in volume. The pellets were washed in 10% NaCl and added to the shrimp isolation containers. The shrimp immediately consumed the pellets, and were fed to satiation. Each shrimp consumed approximately one pellet.

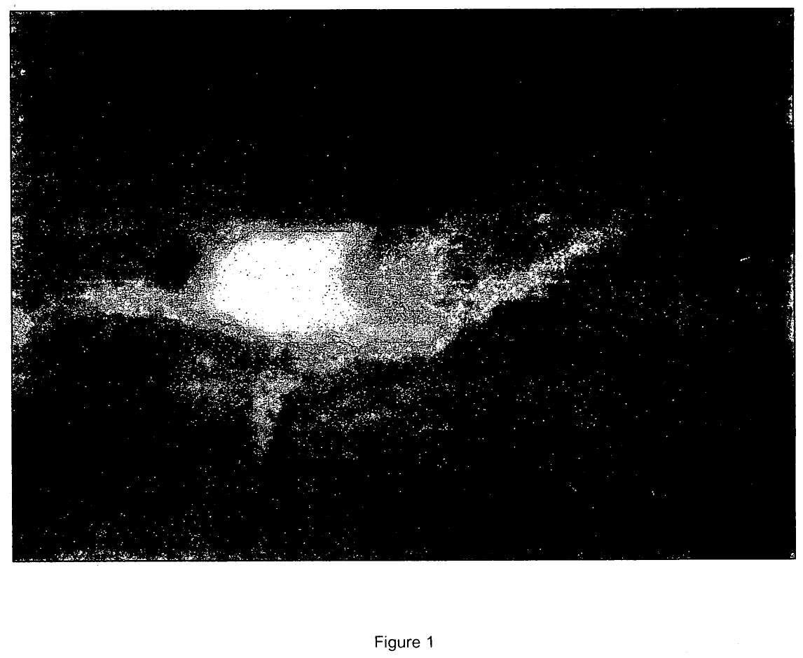

Seventy-two hours after consuming the virally infected matrix, the shrimp were placed in a petri dish and observed on a Dark Reader® transilluminator (Claire Chemical Research). Shrimp expressing GFP exhibited a greenish glow (FIG. 1). Uninfected shrimp demonstrated no fluorescence. The recombinant GFP-tagged baculovirus observed at 72 h was located specifically within the hepatopancreas area in the cephalothorax (FIG. 1).

### Example 8

Vaccination Using Feeds. An antigen characteristic to a particular pathogen is chosen as is required by the animal and circumstances. For example, a viral coat protein or component thereof, or an infectious bacterial protein, or a component thereof is chosen. The gene coding for the protein is isolated and incorporated into a vector suitable for use in the plant or insect of choice for production. The transformation vector is chosen so that the protein will be overexpressed in the algal, plant, animal, arthropod, or insect cell biomass, or in a virus infecting the algal, plant, animal, arthropos, or insect biomass. This biomass is then used as a feed additive in such a way as to provide the viral or bacterial or fungal protein directly to the animal, thus stimulating an immunological response to that particular pathogen. The microbial component may enter the body of the animal in the digestive tract, or otherwise through contact in the air or water.

### REFERENCES

The specification is most thoroughly understood in light of the following references, all of which are hereby incorporated by reference in their entireties.

1. Ausubel, et al., eds. (1987 and periodic supplements) Current Protocols in Immunology, Wiley-Interscience.

2. Bac-to-Bac Baculovirus expression systems manual. Invitrogen Life Technologies. Cat. No. 10359-016.

3. Chalfie, M., Tu, Y., Euskirchen, G., Ward, W. W., and Prasher, D. C. (1994) Green Fluorescent Protein as a Marker for Gene Expression. Science. 263: 802-805.

4. Coligan, et al., eds. (1991 and periodic supplements) Current Protocols in Immunology, Wiley-Interscience.

5. Inoue, S., and Tsuji, F. I. (1994) Aequorea green-fluorescent protein: Expression of the gene and fluorescence characteristics of the recombinant protein. FEBS Letters. 341: 277-280.

6. Lewis, D. L., De Camillis, M. A., Brunetti, C. R., Halder, G., Kassner, V. A., Selegue, J. E., Higgs, S., and Carroll, S. B. (1999) Ectopic gene expression and homeotic transformations in arthropods using recombinant Sindbis viruses. Current Biology 9:1279-1287.

7. Prasher, D. C., Eckenrode, V. K., Ward, W. W., Prendergast, F. G., and Cormier, M. J. (1992) Primary structure of the Aequorea victoria green-fluorescent protein. Gene. 111: 229-233.

1. A feed, feed additive, or therapeutic comprising one or more organisms or any parts thereof, said organism or part thereof comprising one or more proteins, peptides, antibodies, antibody fragments, or combination thereof, which are non-native to the organism and produced by a recombinant virus infecting the organism.

2. The feed, feed additive, or therapeutic of claim 1, wherein the organism is an alga.

3. The feed, feed additive, or therapeutic of claim 1, wherein the organism is an animal.

4. The feed, feed additive, or therapeutic of claim 3, wherein the animal is an arthropod.

5. The feed, feed additive, or therapeutic of claim 4, wherein the arthropod is chosen from a crustacean and an insect.

6. The feed, feed additive, or therapeutic of claim 5, wherein the crustacean is a shrimp.

7. The feed, feed additive, or therapeutic of claim 1, wherein the organism is a plant.

8. The feed, feed additive, or therapeutic of claim 7, wherein the plant is chosen from tobacco, soybean, alfalfa, corn, sunflower, cotton, safflower, and canola.

9. The feed, feed additive, or therapeutic of claim 1, wherein the non-native protein, peptide, antibody, or antibody fragment is specific for bacteria or viruses causing disease in a respective animal chosen from an animal cultivated in water, a farm animal, a ranch animal, a dairy animal, a pet animal, and a human patient.

10. The feed, feed additive, or therapeutic of claim 9, wherein the non-native proteins or peptides are chosen from cecropins, penaeidins, bactenecins, calinectins, myticins, tachyplesins, clavanins, misgurins, pelurocidins, parasins, histones, acid proteins, and lysozymes.

11. The feed, feed additive, or therapeutic of claim 1, wherein the virus is expressed in the organism without incorporation of viral genes into the organism's genome.

12. The feed, feed additive, or therapeutic of claim 11, wherein the virus is an arbovirus.

13. The feed, feed additive, or therapeutic of claim 12, wherein the virus is a Sindbis virus.

14. The feed, feed additive, or therapeutic of claim 11, wherein the virus is a baculovirus.

15. The feed, feed additive, or therapeutic of claim 14, wherein the baculovirus is Autographa californica nuclear polyhedrosis virus.

16. A method of feeding a farm animal, ranch animal, dairy animal, or pet animal comprising administering to said animal a feed comprising an organism, or any part thereof, chosen from algae, plants, arthropods, and other animals, said organism comprising one or more proteins, peptides, antibodies, antibody fragments, or a combination thereof that are non-native to that organism.

17. The method of claim 16, further comprising infecting the organism with a recombinant virus engineered to produce the proteins, peptides, antibodies, or antibody fragments in the organism, and expressing the proteins, peptides, antibodies, or antibody fragments without incorporation of viral genes into the organism's genome.

18. The method of claim 16, wherein the peptide, protein, antibody, or antibody fragment specifically binds to an infectious agent of disease.

19. The method of claim 16, wherein the peptide, protein, antibody, or antibody fragment inhibits the growth or replication of Vibrio.

20. A method of feeding an animal cultivated in water comprising administering to said animal a feed comprising an organism, or any part thereof, chosen from algae, plants, crustaceans, and animals, said organism comprising one or more proteins, peptides, antibodies, antibody fragments, or a combination thereof, that are non-native to that organism.

21. The method of claim 20, further comprising infecting the organism with a virus engineered to produce the peptides, proteins, antibodies, or antibody fragments in the organism, and expressing the proteins, peptides, antibodies, or antibody fragments without incorporation of viral genes into the organism's genome.

22. The method of claim 20, wherein the peptide, protein, antibody, or antibody fragment specifically binds to an infectious agent of disease.

23. The method of claim 22, wherein the peptide, protein, antibody, or antibody fragment inhibits the growth of Vibrio.

24. The method of claim 22, wherein the peptide, protein, antibody, or antibody fragment inhibits viral infection in shrimp.

25. The method of claim 24, wherein the viral infection is caused by Taura virus or White spot virus.

26. The method of claim 16, wherein the recombinant virus is a baculovirus.

27. The method of claim 20, wherein the recombinant virus is a baculovirus.

28. The method of claim 26, wherein the baculovirus is Autographa californica nuclear polyhedrosis virus.

29. The method of claim 27, wherein the baculovirus is Autographa californica nuclear polyhedrosis virus.

30. The method of claim 16, wherein the recombinant virus is an arbovirus.

31. The method of claim 20, wherein the recombinant virus is an arbovirus.

32. The method of claim 30, wherein the recombinant virus is a Sindbis virus.

33. The method of claim 31, wherein the recombinant virus is a Sindbis virus.

34. The method of claim 16, wherein the crustacean is a shrimp.

35. The method of claim 20, wherein the crustacean is a shrimp.
